(19) United States
(12) Patent Application Publication
(10) Pub. No.: US 2017/0281865 A1
(43) Pub. Date: Oct. 5, 2017

(54) Process and system for fluid management during reinjection of adipose tissue

(71) Applicants: John F. Wells, Tucson, AZ (US); McKay S. Crowder, Tucson, AZ (US)

(72) Inventors: John F. Wells, Tucson, AZ (US); McKay S. Crowder, Tucson, AZ (US)

(21) Appl. No.: 15/160,818

(22) Filed: May 20, 2016

Related U.S. Application Data
(63) Continuation-in-part of application No. 15/132,947, filed on Apr. 19, 2016.
(60) Provisional application No. 62/179,885, filed on May 22, 2015.

(51) Int. Cl. A61M 5/172; A61M 5/168; A61M 5/158; A61M 5/14; A61M 5/142

(52) U.S. Cl. CPC A61M 5/172; A61M 5/1413; A61M 5/14228; A61M 5/158; A61M 5/16859; A61M 2005/14208; A61M 2205/3365; A61M 2205/3317; A61M 2205/3306; A61M 2205/3368; A61M 2205/505; A61M 2202/08

(57) ABSTRACT

The invention is a process and system, consisting of a reinjection pump, connecting tubing, a reinjection cannula and a recipient site, with fluid management features including a peristaltic pump head and controls for pressure limits, flow rates and flow distribution wherein the process of controlling the reinjected fat and fluid is unique because of the control of flow rates, pressures, matching cannula hole sizes, and maintaining a closed continuous system where the harvesting and reinjection is done all together with a completely closed system. The process utilizes a method and control system to manage pressure levels during reinjection procedures of viable soft tissue. Although this case specifically relates to its use in the reinjection of adipose fat and other tissue back into the body during liposuction procedures, it can be applied to any medical procedure of introducing or re-introducing materials into the body.

### CROSS REFERENCE TO RELATED PATENT APPLICATION(S)

This application is a non-provisional application being filed under 37 CFR 1.53(b) and 35 USC 111 as a divisional of the presently pending U.S. patent(s) all of which are hereby incorporated by reference.

Application No.—62/179,885

Filing Date—May 22, 2015

Title—Process and System for Fluid Management during Reinjection of Adipose Tissue .

### Background of Invention

A harvesting and reinjection procedure of viable soft tissue typically consists of several different chronological steps [U.S. Pat. No. 8,968,272 Khouri et. al]. including:

1. Infiltration of tumescent fluid

2. Aspiration of viable soft tissue using liposuction

3. Processing aspirated tissue to remove debris, oil, blood, infranate, and saline

4. Reinjecting viable tissue using a cannula

Adipose tissue consists of many small adipocytes held together by fibers in larger agglomerates [U.S. Pat. No. 5,052,999 Klein]. During a liposuction procedure, a harvesting cannula removes these agglomerates, together with other cells and fluids, including saline solution, blood and oil. The harvesting cannula is comprised of a long, hollow metal tube with one or more openings at or near the distal end, and fitted onto a hand piece [U.S. Pat. No. 5,052,999 Klein, Liposuction, Principles and Practice]. The cannula is inserted into a region of the body through a small incision in the skin. With the cannula attached to a vacuum source, adipose tissue is pulled through the openings and into the inner lumen of the cannula [U.S. Pat. No. 5,052,999 Klein, Liposuction, Principles and Practice]. The adipose tissue is then collected in a container [U.S. Pat. No. 8,172,832 Fat harvesting container]

### PRIOR ART

During the previous steps 2 thru 4, viable tissues have been subject to several damaging forces that could compromise their viability[ ]. These forces include:

1. Excessive negative or positive pressures

2. Shearing forces 3. Blunt forces

Liposuction procedures have been performed for decades, and more recently the adipose fat tissue removed has been reinjected into other body parts with moderate success [U.S. Pat. No. 8,968,272 Khouri et. al]. However, very little care was taken to assure tissue viability during the procedure. The focus was to minimize procedure time while maintaining patient safety. Recently, reinjection of harvested tissue has become more popular. This reinjection of adipose tissue has not been an exact science regarding how each of the forces and methods of transfer affect the viability of the live tissue. Individual surgeons base their values and recommendations on experience in doing large numbers of procedures. Patterns emerge when using certain techniques and devices repeatedly, which lead to these values and recommendations. Pressures, flow rates and distribution patterns of reinjected adipose tissue have become some of the important physical characteristics that affect live tissue viability [U.S. Pat. No. 8,968,272 Khouri et. al].

During the aspiration procedure, 18 inHg of negative pressure is considered by some a recommended maximum limit to assure cell viability. Ideally, the negative pressure should only reach workable levels [Wells Johnson Pending Patent ] or that which is only necessary to achieve fat removal; which can vary from patient to patient and will vary from 18 inHg. It has been found that even while pressures required to harvest adipose tissue vary from patient to patient, they can even vary from location to location in the body of the same patient. This means that the surgeon will select the vacuum level(s) required to achieve optimum results from a wide range of vacuum pressures. True fluid management will allow control over a broad range of pressures, match and control those optimum pressures selected during aspiration [U.S. Pat. No. 8,968,272 Khouri et. al and U.S. Pat. No. 8,936,593].

Currently syringes are used for reinjecting the harvested and processed adipose tissue. Once the blood and saline have been removed from the large syringe or container, the processed adipose tissue is typically transferred into a smaller syringe. A reinjection cannula is attached to this smaller syringe and the tissue is reinjected back into the patient using positive pressure. The reinjection cannula has a similar design as the harvesting cannula, consisting of a hollow metal tube with one or more openings at or near the distal end, and fitted onto a hand piece. In the current art, positive pressures are not controlled using this method [Fat harvesting container], and the agglomerates of adipose tissue may experience very high positive pressure as they transition from the large syringe inner diameter to the very small inner diameter of the reinjection cannula. Another disadvantage is that this method requires the use of multiple syringes, with dedicated personnel to fill and handle the syringes.

In the case of a clog, even greater, and most times excessive, positive pressure is applied to clear the clog. A clog occurs when an oversized agglomerate occludes the pathway for adipose tissue. Clogs can be hard to locate, and both time consuming and labor intensive to clear. Currently, clogs are cleared by drastically increasing the positive and/or negative pressures. Positive pressures can reach 40 psi or more using a syringe [U.S. Pat. Nos. 5,002,538, 8,771,592]. This is well in excess of the range of pressures used during aspiration. While the reinjection procedure is done using a syringe, in some cases a peristaltic pump may be used. Current peristaltic pumps have only settings for in reinjection can easily achieve 25 psi or more of pressure which is also well in excess of the range of pressures used during aspiration.

During an aspiration procedure, controlling flow rates and pressures is a very common and standard practice, however very little attention is given to the reinjection side of the procedure in regards to flow rates and pressures. A vacuum level can never exceed minus 1 atm, so regardless of how fast the flow rate is this level will never be exceeded. Flow rates and pressures are more loosely tied in an aspiration procedure, since a custom vacuum level can be set using a regulator or relief valve near the pumps. This ensures that the vacuum level never exceeds that amount anywhere upstream of the regulator, regardless of how high the flow rate of the vacuum pumps. With reinjection, positive pressures are used to move the adipose tissue, and extreme pressures of well over 1 atm can be easily achieved. This requires closer control of both flow rates and pressures when reinjecting adipose tissue. The ideal goal will be to maximize flow rates, while at the same time controlling the positive pressure levels in the tubing, the reinjection cannula and the recipient site.

### BRIEF SUMMARY AND NOVELTY OF INVENTION

The invention is a process and a system that is all inclusive of a system that includes all parts labeled in the five figures and the process that utilizes any combination of the current features not found in the prior art. The novelty of this process includes the simultaneous control of the reinjection pressure and flow rates while matching the holes' sizes in the reinjection cannula with those of the harvesting during the same process or procedure of removing the fat cells and fluid.

While flow rates and pressures are important, the distribution and pattern of the flow plays a role in tissue viability. It has been recommended that agglomerates of adipose tissue not be too large, and to be spread out. This is because after the agglomerate has been reinjected to the recipient site of a patient, each adipocyte must receive a blood supply to survive. Without a blood supply, the adipocyte dies and is recycled by the body. With an agglomerate that is too large, or with too many agglomerates stuffed into one area, blood flow can be restored only to the adipocytes on the outer surface, while the adipocytes located deep inside the agglomerates do not receive blood. This causes a collapse of the agglomerate as the adipocytes die, and results in unpredictable long term results of the reinjection procedure.

The flow distribution pattern is traditionally done by pressing the syringe plunger using ones thumb. By moving in a constant motion, pushing and releasing the plunger in a pattern creates boluses. Another method is to apply constant pressure to the plunger and make small stop and go motions with the hand as the cannula is moved back and forth through the recipient site. Both of these methods can create smaller, segmented boluses inside the recipient site. An improvement of this style is to automate the creation of these boluses using the reinjection pump. No such feature is available with current peristaltic pumps, so the pattern cannot be duplicated. This automation of pulses would allow the surgeon to move the reinjection cannula steadily in a continuous motion, while the pump creates the pulses and boluses.

What is needed is a system to allow fluid management that is precise and easy to use which allows control over pressure levels, flow rates and flow distribution patterns during the reinjection procedure.

### SUMMARY OF THE INVENTION:

The preferred embodiment of the present invention is the system and process of reinjecting adipose tissue with fluid management controls. The reinjection process of the present invention comprises the following:
1. having a container with harvested and processed adipose tissue
2. a reinjection unit with a motorized peristaltic pump with control features and a touch screen user interface
3. flexible connecting tubing
4. a reinjection cannula
5. a recipient site

A preferred embodiment of the invention and process is as follows. The reinjection pump uses a peristaltic pump head, along with flexible tubing to move adipose tissue from the container, through the reinjection cannula and into the recipient site. One end of the flexible tubing is attached to the container. The tubing is run through the peristaltic pump head and an external sensor. When the pump is activated, it draws the material from the container using a vacuum force. Once the material passes through the pump head it is moved using positive pressure. The external sensor measures this internal positive pressure and provides feedback to the reinjection pump. The feedback allows the user to control an upper limit for this pressure, as well as pumped through the tapered entrance of the reinjection cannula and into the recipient site. If at any time the pressure exceeds the limit set by the user, the pump automatically stops and reverses to zero pressure. A footswitch is installed to give remote access to the on/off function of the pump.

When all the above parts of the preferred embodiment of the present invention are working together, the following features and benefits may be achieved:
1. Peristaltic pump for reinjection. This type of pump has many special benefits over all the other types of pumping systems:
   a. The fluid does not make direct contact with the pump head or components, it stays within the inner lumen of the flexible tubing. This minimizes the sterilization to only disposable flexible tubing.
   b. The pump head pulls a vacuum on one side and creates positive pressure on the other. This eliminates the need for any type of valves in the tubing system or pump.
   c. The positive displacement pumping action creates a gentle movement of the live tissue through the tubing.
   d. The peristaltic pump uses one continuous piece of tubing, eliminating any transitions or lumen diameter changes.
2. An external pressure sensor. This invention includes a novel design of measuring positive pressure of fluid in the flexible tubing by way of clamping to the outside of the tubing with a transducer, wherein the preferred transducer is a load cell.
3. Control of pressures, flow rates and patterns of fluid flow in the flexible tubing.
4. Reinjection cannula has a tapered entrance to allow a more gradual transition between the large inner diameter of the flexible tubing and the smaller inner lumen of the reinjection cannula, thus reducing pressure spikes, clogs, and possible over-pressure situations.

The preferred embodiment of the present invention allows controls of pressure, flow speeds and flow patterns. Using a mechanical pump to reinject adipose tissue can be dangerous to the patient and to the adipocytes without controls over important parameters. Pressure is defined as the positive pressure developed in the flexible tubing after it has been pushed through the pump. The present invention measures the pressure using an external pressure sensor that clamps onto the outside of the flexible tubing. The preferred embodiment uses a load cell that measures force. The internal pressure expands/contracts the flexible tubing and exerts a force on the load cell that is proportional to the pressure. Flow speeds are adjusted by simply changing the speed with which the pump moves, whether rotational or linear. Flow distribution pattern settings are controlled by indexing the pump into small increments of stop and go motion. The frequency of this stop-and-go motion can be adjusted up or down and relates to the number of pulses per minute. The size of the pulse is adjusted by increasing or decreasing the speed of the rotation during each stop and go motion.

The preferred embodiment of monitoring the positive internal pressure of the tubing is described above, however measuring this internal positive pressure is not limited to that only. A sensor may be used to detect internal pressure from outside or inside the flexible tubing, which may be a transducer of any of the following types: piezoresistive strain gauge, capacitive, electromagnetic, piezoelectric, optical, from the transducer is to allow a closed loop monitoring of pressure to the reinjection pump, however the signal from the transducer may also be used as an open loop monitoring. An example would be that the signal from the transducer would be used to display a live readout of the pressure to the user. Another way to monitor pressure in the tubing is to monitor the motor itself. Increasing pressure may be translated as an increased load on the motor. This increased load may be monitored using a sensor for current, voltage, torque, force or any combination of two or more of these.

### BRIEF DESCRIPTION OF THE FIGURES

FIG. 1: A basic overview of the entire reinjection system

FIG. 2: A perspective overview of the reinjection pump

FIG. 3: A front view of the peristaltic pump head action with tubing installed

FIG. 4a: A side view representation of the open external sensor with tubing

FIG. 4b: A side view representation of the closed external sensor with tubing

FIG. 5: A graphical representation of the flow pattern feature.

FIG. 6: A pictorial diagram and cross-sectional of a typical cannula

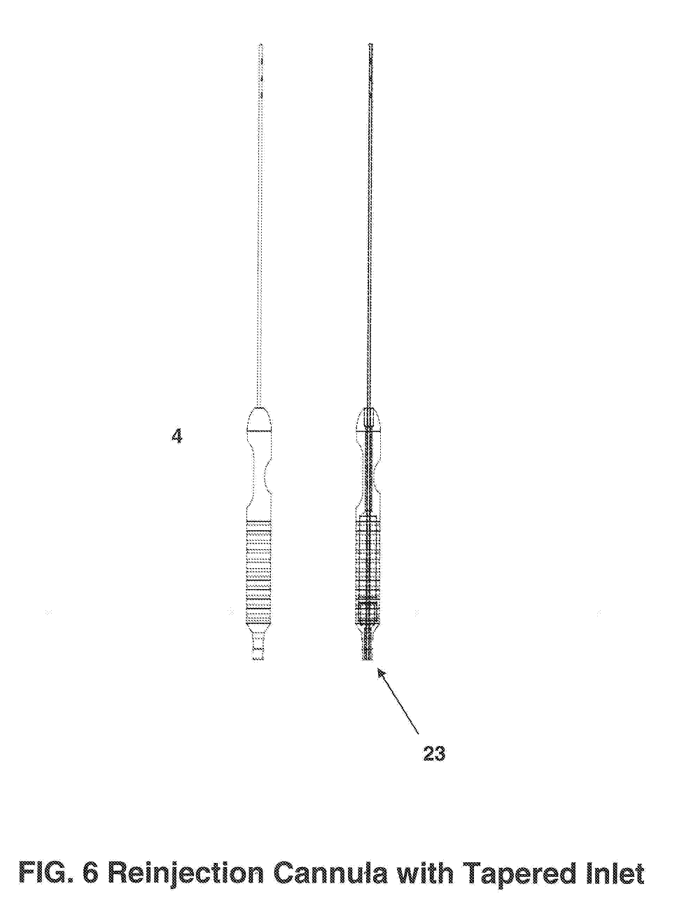

### DETAILED DESCRIPTION OF THE INVENTION

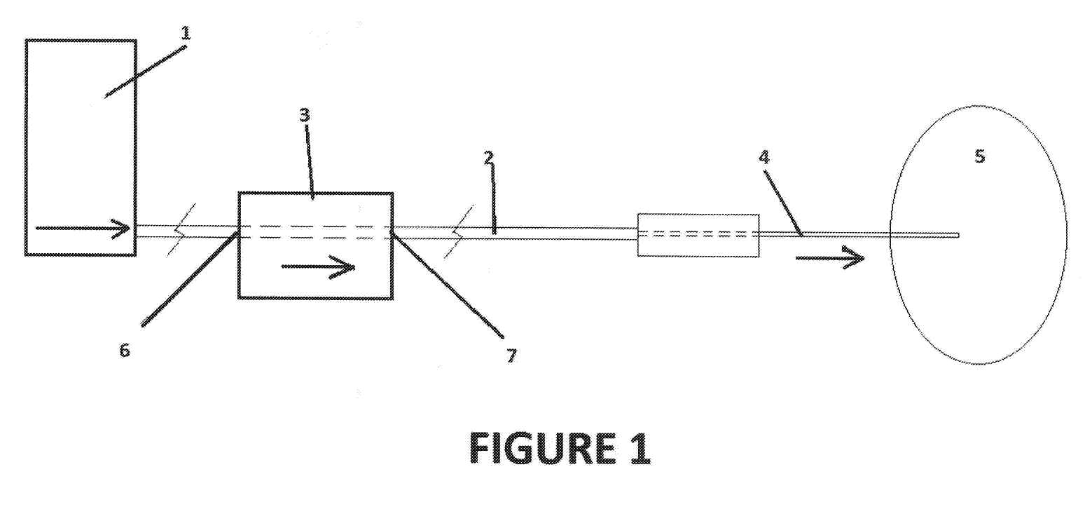

The present invention consists of a process and system of parts that together form a reinjection system and process for transferring adipose tissue and providing fluid management. The process and system of parts consists of a canister or container (1), reinjection pump (3), flexible tubing (2), a reinjection cannula (4) and a recipient site (5). It includes the specific novel features of this process not previously utilized in the prior art. FIG. 1 shows an overview of the setup of the apparatus. Adipose tissue that has been harvested is collected in a container (1), which will have a port or entry to allow the adipose tissue to be drawn out. One end of the flexible tubing (2) is attached to the port of the container while the flexible tubing (2) is run through the reinjection pump (3). A reinjection cannula (4) is connected to the other end of the flexible tubing (2), and is used to reinject adipose tissue to the recipient site (5). The container (1) is located on the negative pressure side of the pump (6), in which the negative pressure draws adipose tissue into the flexible tubing (2). The positive pressure side of the pump (7) is located after the reinjection pump (3) and is what pushes the adipose tissue through the flexible tubing (2), the reinjection cannula (4), and ultimately into the recipient site (5). The recipient site (5) includes the patient, however it encompasses anywhere that the adipose tissue goes when it leaves the reinjection cannula (4).

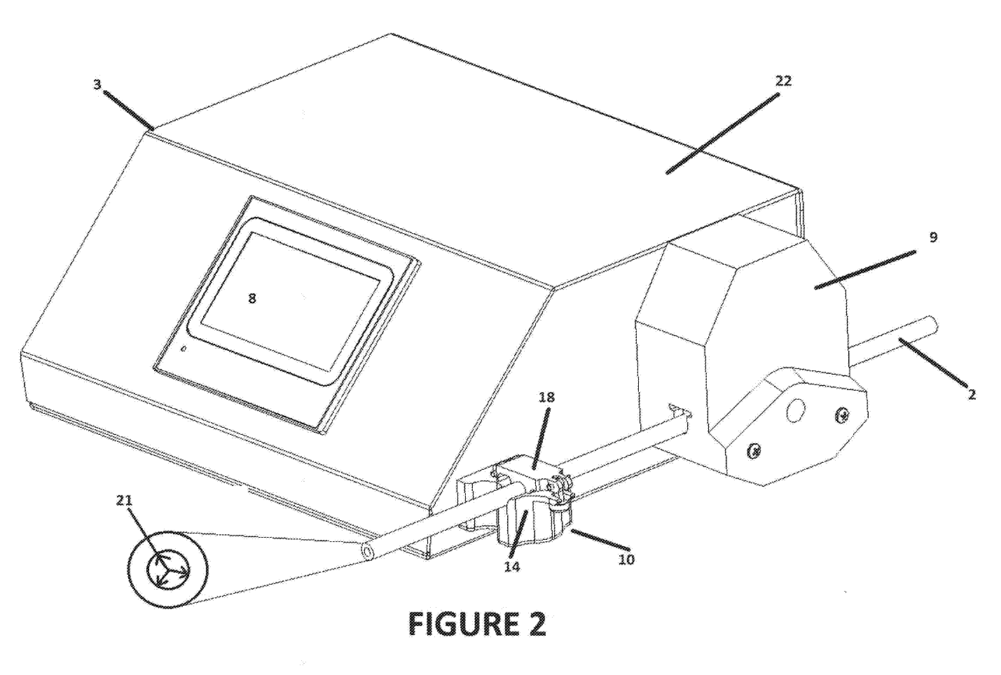

FIG. 2 shows the perspective view of the reinjection pump (3). It has an enclosure (22), typically made from multiple pieces of formed metal that are fastened together, but it can be made from any kind of molded plastic or polymer. This enclosure (22) holds all the power components that make up the reinjection pump (3), as well as the major components. These include the peristaltic pump head (9), the external mounted to a DC motor that can rotate in both clockwise and counter clockwise directions. The external pressure sensor (10) is made of several components, of which the base (14) may be permanently mounted to the enclosure (22), or it may be free floating. Either way, the purpose is to monitor the internal pressure (21) in the tubing and provide feedback to the reinjection pump (3). The sensor for measuring pressure may even be a free standing device that is separate to the reinjection pump (3), however this is still under the patented idea as long as the freestanding device provides feedback of internal pressure (21) to the reinjection pump (3) in the form of an electrical signal. The touch screen (8) allows the user to set variables and settings of the reinjection pump (3). These settings include, but are not limited to the revolutions per minute of the motor, the pressure limit, and the number of pulses per minute.

The setting for revolutions per minute is referred hereafter as the speed of the motor. The speed is also the revolutions per minute of the peristaltic pump head (9), since the motor shaft is directly coupled to the peristaltic pump head (9) shaft. Control of the speed is very important for reinjecting adipose tissue because the tissue can only flow so fast through the flexible tubing (2) and the reinjection cannula (4). The main flow restriction is the small inner lumen of the reinjection cannula (4). If the speed setting is too high, the adipose tissue will be pushed into the flexible tubing (2) faster than it is leaving. This will increase the pressure and may trigger the upper pressure limit of the reinjection pump (3). The reinjection cannula (4) has a machined taper (23) where the flexible tubing (2) connects to the reinjection cannula (4). This taper (23) acts as a down to the smaller inner diameter of the reinjection cannula (4).

The pressure limit of the reinjection pump is the maximum internal pressure (21) desired inside the flexible tubing (2). If the external pressure sensor (10) detects a pressure that exceeds the limit, it will immediately stop the motor, which begin pumping in reverse until the internal pressure (21) is reduced to near zero. The zero pressure is simply the internal pressure (21) when the sensor is zeroed before the procedure, and is typically atmospheric pressure. The user will have to release and press the footswitch again to resume normal forward pumping. The pressure limit feature using the external pressure sensor (10) automatically limits the pressure, and allows the surgeon to focus on more important matters, such as placement of reinjected tissue.

The ability to set a number of pulses per minute allows the user to create a pattern to the flow. Pulses are created by rotating the peristaltic pump head (9) forward quickly a small amount, stopping, and moving forward again quickly a small amount. When set correctly, the pulses propagate through the fluid in the flexible tubing (2), with the effect that the fluid exits the end of the reinjection cannula (4) in small boluses, or droplets, automatically. This can eliminate the back and forth motion of the hand during reinjection by automatically placing a segmented array of boluses with a straight movement of the cannula end. Controlling the bolus size is an important factor in maintaining adipose tissue viability by spacing out the agglomerates and maximizing vascularization.

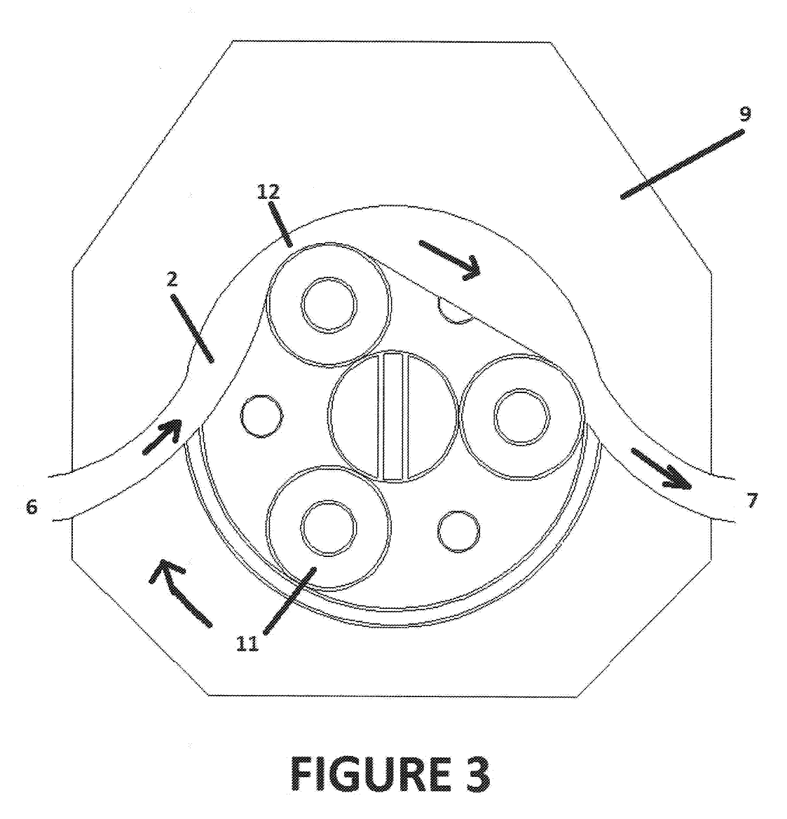

FIG. 3 shows a side view of the peristaltic pump head (9) with the flexible tubing (2) installed. The peristaltic pump uses positive displacement to move material, and this is created using multiple rollers (11). As the DC motor turns the rollers (11), a roller pushes the tubing against the surface of the pump head creating a seal by compression (12). As the roller moves past the compressed area, the flexible tubing (2) goes back to its original shape. This creates the negative pressure side of the pump (6) and draws in more fluid. The fluid trapped before the compression (12) point is forced forward and creates a positive pressure side of the pump (7). Before the first roller releases the compression (12) section, the roller behind it initiates a new compression (12) section and the process repeats. Thus the rollers (11) act as both the pumping mechanism, and a valve to separate the positive and negative pressure sides.

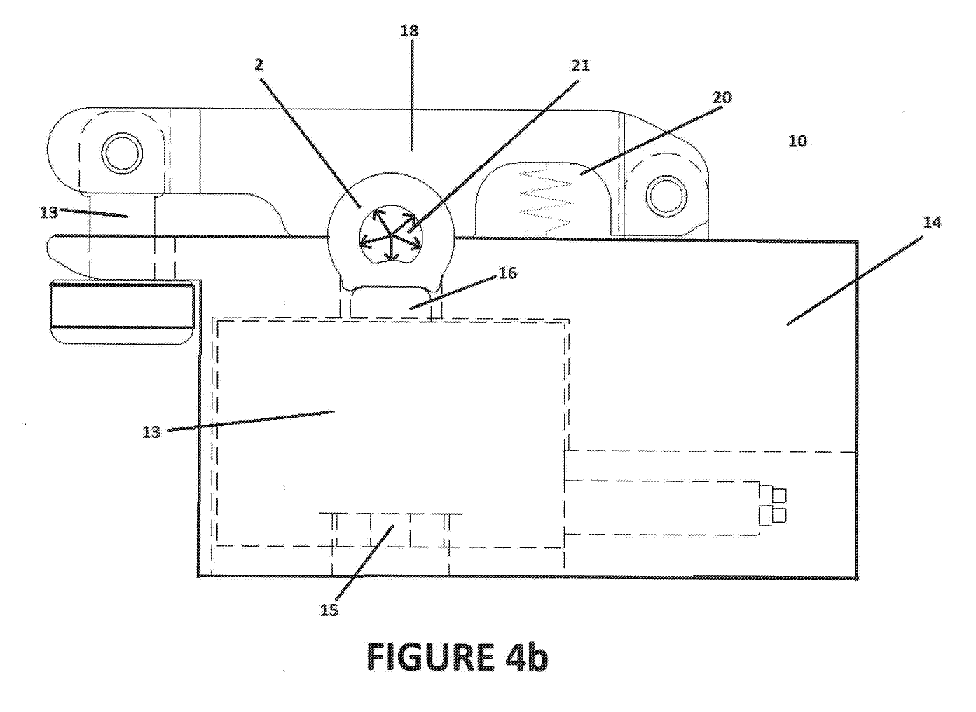

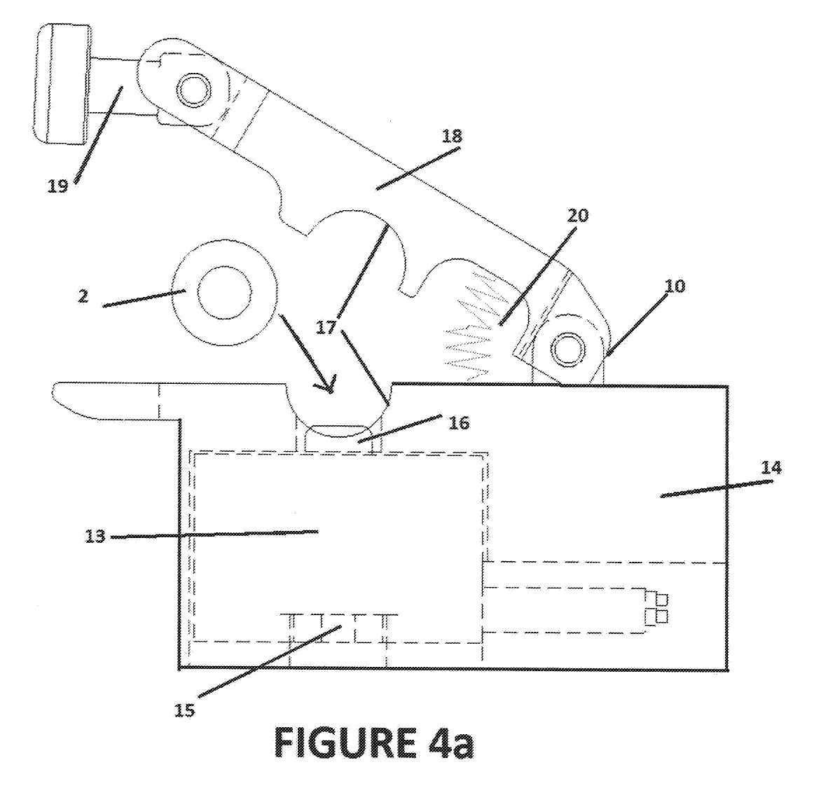

FIG. 4a shows a side view of the preferred embodiment of the external pressure sensor (10). When the locking mechanism (19) is released, the top hinge (18) opens to allow the tubing to be installed. Both the base (14) and the top hinge (18) have a tubing channel (17) that matches the outer diameter of the flexible tubing (2). The transducer/load cell (13) is rigidly attached (15) to the base (14) with the sensing component (16) of the transducer/load cell (13) protruding into the tubing channel (17). The flexible tubing (2) is placed into the tubing channel (17), the top hinge (18) is closed, and the locking mechanism (19) is engaged. The locking mechanism (19) simply prevents the top hinge (18) from moving and creates a rigid support for the flexible tubing (2) to press against. The spring (20) is used simply to hold open the top hinge (18) when the locking mechanism (19) is not engaged. The spring (20) also provides a force to keep the locking mechanism (19) engaged when the external pressure sensor (10) is closed with no flexible tubing (2).

FIG. 4b shows the same side view as FIG. 4a, but with the external pressure sensor (10) in the closed position with flexible tubing (2) installed. This figure shows a representation of the deformation of the flexible tubing (2) when it is pressed against the sensing component (16). With the transducer/load cell (13) rigidly attached (15) to the base (14), the only part that can move is the sensing component (16) as it deforms the internal structure of the transducer/load cell (13) containing the strain gauge. The flexible tubing (2) is confined by the tubing channel (17) and will not expand in any direction except towards the sensing component (16). This ensures that the expansion of the flexible tubing (2) from the force caused by the internal pressure (21) is focused towards the sensing component (16). This force, which is defined as a force over a specific area, varies directly with the internal pressure (21) in the tubing, and thus an accurate relationship is made between the electrical signal from the transducer/load cell (13) and the internal pressure (21). A major benefit to this sensor is that it is external, and does not need any direct contact with the adipose tissue inside. This makes maintaining sterility very easy, and the entire external pressure sensor (10) is reusable. Reusability increases the accuracy and ease of use by eliminating repeated calibration steps.

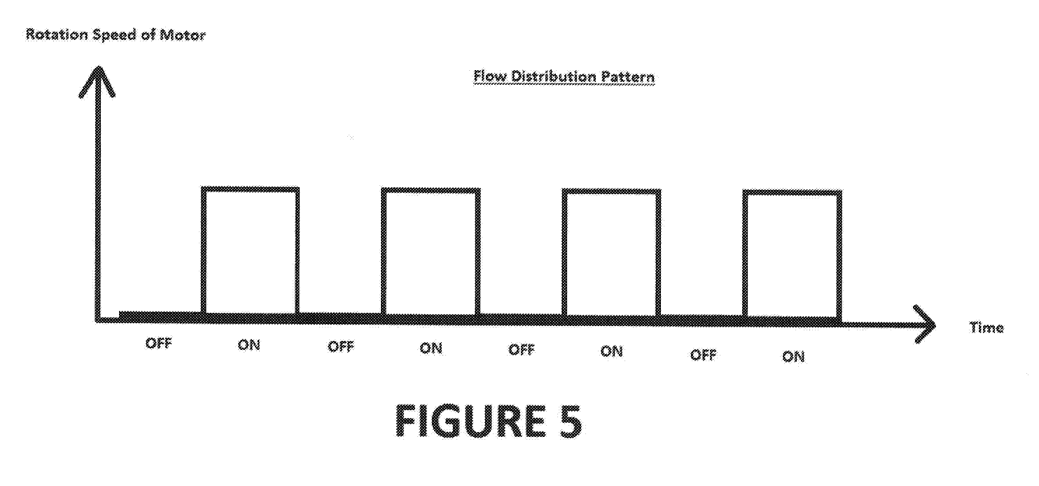

FIG. 5 shows a representation of the flow distribution pattern and how it is controlled using the pulse feature. The pulses can be set using two variables, the rate of the stop and go motion, and the speed of the motor during each go motion. The purpose of the pulse function is to create separate, segmented boluses of adipose tissue that exit the reinjection cannula (4). The rate of the stop and go motion can be described as the number of pulses per minute, and can be attributed to the number of droplets per minute. The amount of rotation during each pulse affects the size of each droplet. A higher speed means the motor rotates farther during each pulse, and pushes a larger volume of fluid each time. This type of flow distribution is important to maximize vascularization to the adipose tissue. Distributing the agglomerates of adipose tissue by spacing them out gives each agglomerate the largest chance of finding a blood supply.

The present invention includes a focus on maintaining a constant, smooth, continuous lumen throughout the entire system. This means that after the adipose tissue leaves the container (1), the inner diameter of the flexible tubing (2) and all connections are to be maintained through all processes, until it reaches the reinjection cannula (4). This helps the adipose tissue move gently along the flexible tubing (2) path by removing choke points and small orifices to squeeze through, such as luer lock fittings and one way valves. The system contains only one component that has an active role in contacting the adipose tissue, the flexible tubing (2). This flexible tubing (2) comes in disposable, pre-sterilized packs to allow a sterile environment for the adipose tissue.

What is claimed is:

1. A process and system of parts that make up a system to transfer adipose tissue with fluid management features, comprising the following:
a. a container for the harvested adipose tissue, with an adequate port for removing the adipose tissue using the reinjection pump and flexible tubing
b. a reinjection pump, which uses a peristaltic pump head to pump adipose tissue and a touch screen interface for controls
c. flexible tubing creating fluid communication between a container, and the reinjection cannula.
d. a reinjection cannula, comprised of a long, hollow metal tube with one or more openings at or near the distal end, fitted onto a hand piece, and with a tapered transition point where the flexible tubing attaches to the cannula.
e. a recipient site, which is anywhere the adipose tissue goes when it leaves the reinjection cannula.
f. a sensor for providing feedback of internal pressure in the flexible tubing to the reinjection pump, wherein the sensor is attached to a housing that clamps around the flexible tubing, such that the sensor is held firmly against the outside of the flexible tubing, and such that the sensor is a load cell.
g. a live readout of the internal pressure is displayed on the touch screen interface
h. wherein the sensor housing has a quick release locking mechanism to allow easy, quick access for installing/uninstalling the flexible tubing.
i. where one or more component(s) of the housing of the sensor of claim 1e is/are permanently attached to the reinjection pump enclosure.
j. where the reinjection pump allows for control of motor speed, typically in revolutions per minute.
k. where the reinjection pump allows for control of flow rates.
l. the internal pressure may be set as an upper limit by the user on the reinjection pump.
m. the flow distribution pattern is set by the user using controls on the reinjection pump.

2. The process and system of parts of claim 1 wherein one or more component(s) of the housing of the sensor of claim 1e is/are free floating, while still allowing the firm contact of the sensor to the flexible tubing.

3. A process and system of parts that make up a system to transfer adipose tissue with fluid management features, comprising the following:
a. a container for the harvested adipose tissue, with an adequate port for removing the adipose tissue using the reinjection pump and flexible tubing
b. a reinjection pump, which uses a peristaltic pump head.
c. flexible tubing creating fluid communication between a container, and the reinjection cannula.
d. a reinjection cannula, comprised of a long, hollow metal tube with one or more openings at or near the distal end, and fitted onto a hand piece, and with a tapered transition point where the flexible tubing attaches to the cannula.
e. a recipient site, which is anywhere the adipose tissue goes when it leaves the reinjection cannula.
f. a sensor for providing feedback of internal pressure in the flexible tubing to the reinjection pump, such that the sensor is a transducer of any of the following types: piezoresistive strain gauge, capacitive, electromagnetic, piezoelectric, optical, potentiometric, resonant, thermal, or ionization.
g. a live readout of the internal pressure is displayed on the touch screen interface
h. where the reinjection pump allows for control of motor speed, typically in revolutions per minute.
i. where the reinjection pump allows for control of flow rates.
j. the internal pressure may be set as an upper limit by the user on the reinjection pump.
k. the flow distribution pattern is set by the user using controls on the reinjection pump.

4. A process and system of parts that make up a system to transfer adipose tissue with fluid management features, comprising the following:
a. a container for the harvested adipose tissue, with an adequate port for removing the adipose tissue using the reinjection pump and flexible tubing
b. a reinjection pump, which uses a peristaltic pump head.
c. flexible tubing creating fluid communication between a container, and the reinjection cannula.
d. a reinjection cannula, comprised of a long, hollow metal tube with one or more openings at or near the distal end, and fitted onto a hand piece, and with a tapered transition point where the flexible tubing attaches to the cannula.
e. a recipient site, which is anywhere the adipose tissue goes when it leaves the reinjection cannula.
f. a sensor for providing feedback of internal pressure in the flexible tubing to the reinjection pump, wherein the motor is monitored for changes in loading based on changing pressures in the flexible tubing, such that the increased load of the motor is monitored using a sensor for current, voltage, torque, force or any combination of two or more of these.
g. a live readout of the internal pressure is displayed on the touch screen interface
h. where the reinjection pump allows for control of motor speed, typically in revolutions per minute.
i. where the reinjection pump allows for control of flow rates.
j. the internal pressure may be set as an upper limit by the user on the reinjection pump.
k. the flow distribution pattern is set by the user using controls on the reinjection pump.

5. The flow distribution of any of the preceding claims wherein the frequency of the stop and go motion and the speed of rotation of the motor are set by the user through the reinjection pump.

6. The recipient site of any of the preceding claims wherein the recipient site is a location(s) in the patient undergoing the reinjection procedure.

7. The process and system of parts of any of the preceding claims wherein the flexible tubing is made from one continuous piece.

8. The tubing of claim 7 wherein the peristaltic pump is able to pump on any section of the tubing.

9. The tubing of claim 7 wherein the external transducer for measuring internal pressure can be sensed on any section of the tubing.

10. The process and system of parts of any of the preceding claims wherein the flexible tubing is made from one or more sections of tubing.

11. The tubing of claim 10 wherein each section may vary in material, diameter and size.

12. The tubing of claim 10 wherein the peristaltic pump head is required to pump on a specific section of the tubing.

13. The tubing of claim 10 wherein the external transducer for measuring internal pressure is required to be sensed on a specific section of the tubing.

14. The pressure limit of any of the preceding claims wherein the monitoring of internal pressure extends to the recipient site, since any given internal pressure of the flexible tubing is also the pressure of the fluid just exiting the cannula into the recipient site.

15. The pressure monitoring of claim 14 wherein the surgeon may control, observe and gain insight into the pressure at the recipient site through the live readout of pressure displayed by the reinjection pump, including interstitial pressure of the recipient site, tissue type of the recipient site, overfilling of adipose tissue due to holding the reinjection cannula in one place too long, patient discomfort from pressure limit too high.
